(19) United States
(12) Patent Application Publication
(10) Pub. No.: US 2021/0244561 A1
(43) Pub. Date: Aug. 12, 2021

(54) VARIABLE DENSITY ORTHOSIS

(71) Applicant: KINTEC FOOTLABS INC., Surrey (CA)

(72) Inventor: Michael Ryan, Surrey (CA)

(21) Appl. No.: 17/049,451

(22) PCT Filed: Apr. 9, 2020
(86) PCT No.: PCT/CA2020/050479

§ 371 Date: Oct. 21, 2020

Related U.S. Application Data
(60) Provisional application No. 62/832,391, filed on Apr. 11, 2019.

(51) Int. Cl. A61F 5/14; A43B 7/14

(52) U.S. Cl. CPC A61F 5/14; A43B 7/141

(57) ABSTRACT

There is provided an orthosis that includes an insole having a lateral side portion and a medial side portion. The medial side portion is formed from a first material having a first density and the lateral side portion formed from a second material having a second density, wherein the second density is greater than the first density. The medial side portion and the lateral side portion join at an oblique angle. In addition, the medial side portion of the insole has a contoured arch support portion. In some embodiments, the orthosis further includes a lateral wedge coupled to a bottom of the insole, wherein the lateral wedge having an opposite side thereof positioned at a lateral side of the orthosis.

### FIELD OF THE INVENTION

The present invention pertains to the field of orthoses and in particular to an apparatus for alleviating medial knee compartment loading, for example which may alleviate knee osteoarthritis.

### BACKGROUND

It is known that osteoarthritis (OA) is a prevalent form of arthritis. Knee OA and its associated risk of mobility disability, which may include requiring assistance walking or climbing stairs, can be a greater problem for people ≥65 than other medical conditions. As such the societal burden, for example in terms of personal suffering and use of health resources, is expected to increase with the increasing prevalence of obesity and the ageing of the community.

Common symptoms of knee OA include pain, instability as well as loss of muscle power and range of motion, each of which can make everyday activities more difficult. These everyday activities can include action like rising from a chair and walking. Knee OA is considered a degenerative condition often treated with pain medications or in more severe cases, total joint replacement.

Knee bracing is a common conservative non-medicinal treatment to ‘unload’ the medial compartment of the knee by decreasing the knee adductor moment, namely the force that pushes the knee into a bow-legged position. While effective, knee braces can be costly and skin irritation can be a common skin-effect. It has been seen that many patients avoid bracing altogether due to cosmetic concerns and the perception of frailty.

Another non-medicinal approach is shoe-based lateral wedges, where the lateral or outside edge of the insole is elevated relative to the medial or inside edge of the foot. This approach has also been used by the podiatry and pedorthic industries to create a similar degree of joint unloading as compared to knee bracing, while also overcoming the cost, cosmetics, and skin irritation challenges associated with knee bracing. However, it has been found that lateral wedges can place the foot and ankle in a less stable position. As such lateral wedges are currently seldom used for knee OA given concerns about comfort and secondary injury that the lateral wedge may cause.

In Human Movement Science, August 2013; pages 596-604, “The biomechanical effects of a new design of lateral wedge insole on the knee and ankle during walking”, Richard K. Jones, Min Zhang, Philip Laxton Andrew H. Findlow and Anmin Liu disclosed that the concept of combining a lateral wedge with contoured arch support, which was demonstrated as a design that was able to both stabilize the ankle and unload the medial compartment of the knee. However, in the British Journal of Sports Medicine, February 2018, pages 238-252, titled “The effects of shoe-worn insoles on gait biomechanics in people with knee osteoarthritis: a systematic review and meta-analysis” it was concluded that current versions of the ‘arch-supported’ lateral wedge minimizes unloading effects at the knee, wherein the unloading of the medial compartment of the knee is desired to alleviate the effects of knee OA.

Accordingly, there may be a need for an apparatus for alleviating medial knee compartment loading, which may for example alleviate knee osteoarthritis that is not subject to one or more limitations of the prior art.

This background information is intended to provide information that may be of possible relevance to the present invention. No admission is necessarily intended, nor should be construed, that any of the preceding information constitutes prior art against the present invention.

### SUMMARY

It is an object of the present invention to obviate or mitigate at least one disadvantage of the prior art.

According to another aspect of the present invention there is provided an orthosis. The orthosis includes an insole having a lateral side portion and a medial side portion. The medial side portion is formed from a first material having a first density and the lateral side portion formed from a second material having a second density, wherein the second density is greater than the first density. The medial side portion and the lateral side portion join at an oblique angle. In addition, the medial side portion of the insole has a contoured arch support portion.

In some embodiments, the orthosis further includes a lateral wedge coupled to a bottom of the insole, wherein the lateral wedge having an opposite side thereof positioned at a lateral side of the orthosis. In some embodiments, the orthosis further includes an arch support material infilling an arch volume formed by the contoured arch support portion. In some embodiments, the orthosis further includes a contoured lateral portion and a contoured posterior portion.

According to an aspect of the present invention, there is provided a method for making an orthosis. The method includes forming a medial side portion of an insole from a first material having a first density, the medial side portion further having a contoured arch support portion. The method further includes forming a lateral side portion of an insole from a second material having a second density, the second density greater than the first density. In addition, the method includes joining the medial side portion and the lateral side portion, wherein the medial side portion and the lateral side portion join at an oblique angle along a length of the insole.

In some embodiments, the method further includes attaching a lateral wedge to a bottom of the insole, the lateral wedge having an opposite side thereof positioned at a lateral side of the orthosis. In some embodiments, forming one or more of the medial side portion, the lateral side portion and the lateral wedge is performed using three dimensional printing.

Embodiments have been described above in conjunction with aspects of the present invention upon which they can be implemented. Those skilled in the art will appreciate that embodiments may be implemented in conjunction with the aspect with which they are described but may also be implemented with other embodiments of that aspect. When embodiments are mutually exclusive, or are otherwise incompatible with each other, it will be apparent to those skilled in the art. Some embodiments may be described in relation to one aspect, but may also be applicable to other aspects, as will be apparent to those of skill in the art.

### BRIEF DESCRIPTION OF THE FIGURES

Further features and advantages of the present invention will become apparent from the following detailed description, taken in combination with the appended drawings, in which:

FIG. 1 illustrates a perspective view of a variable density orthosis for a right foot according to embodiments of the present invention.

FIG. 2A illustrates a posterior view of a first embodiment of the variable density orthosis illustrated in FIG. 1.

FIG. 2B illustrates a posterior view of a second embodiment of the variable density orthosis illustrated in FIG. 1.

FIG. 2C illustrates a posterior view of a third embodiment of the variable density orthosis illustrated in FIG. 1.

FIG. 2D illustrates a posterior view of a fourth embodiment of the variable density orthosis illustrated in FIG. 1.

FIG. 3A a top view of a variable density orthosis for a right foot including a contoured shell according to embodiments of the present invention.

FIG. 3B a top view of a variable density orthosis for a right foot including a contoured shell according to embodiments of the present invention.

FIG. 4A a perspective view of a variable density orthosis for a right foot including a textured top surface according to embodiments of the present invention.

FIG. 4B illustrates a cross sectional view of a texture formation according to embodiments of the present invention.

FIG. 5 illustrates a method an orthosis for alleviating osteoarthritis according to embodiments of the present invention.

### DETAILED DESCRIPTION

It has been realised that providing a variable density orthosis can be able to create a similar degree of medial compartment unloading of the knee as is provided by a lateral wedge, while also improving comfort and limiting a corresponding increase in ankle instability, when compared to a lateral wedge.

According to embodiments, the unloading effect of the knee by way of a lateral wedge is provided by use of a density differential in the material used to form the orthosis, wherein the orthosis further provides a level of stabilization of the ankle through a contoured arch support.

According to embodiments, the unloading of the medial knee compartment can aid with the alleviation of conditions which may include one or more of osteoarthritis of the knee, medial meniscal injuries, peroneal tendon injuries and lateral ankle instability. Other conditions that may benefit from the unloading of the medial knee compartment would be readily understood by a worker skilled in the art.

There is provided an orthosis that includes an insole having a lateral side portion and a medial side portion. The medial side portion is formed from a first material having a first density and the lateral side portion formed from a second material having a second density, wherein the second density is greater than the first density. The medial side portion and the lateral side portion join at an oblique angle. In addition, the medial side portion of the insole has a contoured arch support portion. In some embodiments, the orthosis further includes a lateral wedge coupled to a bottom of the insole, wherein the lateral wedge having an opposite side thereof positioned at a lateral side of the orthosis.

According to embodiments, the contoured arch support can be custom made for an individual, for example through the creation of a 3D foot impression. In other embodiments, the contoured arch support can be selected for an individual based on a generic arch shape. The generic arch shape may divided into multiple versions thereof, which can account for a level of variability in arches of different individuals. For example, the generic arch shape can have arch height versions available to accommodate high, medium, or low arched foot postures. The arch support can provide a level of stabilization of the foot and ankle and can balance the effect of the further features of the orthosis which are configured to the unloading of the knee.

According to embodiments, medial side portion of the orthosis, for example the portion of the orthosis to be positioned under the inner side of the foot, is composed of a material having a first density. The lateral side portion of the orthosis, for example the portion of the orthosis to be positioned under to the outer side of the foot, is composed of a material having a second density, wherein the second density is greater than the first density. The ratio of these densities can vary. According to some embodiments, the lateral side portion and the medial side portion of the orthosis join at approximately the midline of the long axis of the insole. However it would be readily understood that this joining location of the lateral side portion and the medial side portion can be offset of the midline of the long axis either towards the lateral side or the medial side of the orthosis.

According to embodiments, density ratio can be a 75:25 durometer ratio for the lateral side portion and the medial side portion respectively. While this may be a desired density ratio for the orthosis, other ratios are possible, for example 80:20, 90:10, 60:40 or other ratio as would be readily understood.

In some embodiments the orthosis is formed from of ethyl vinyl acetate (EVA). However, the composition of the material is not limited to EVA but could include any open- or closed-cell foam, including plastizotes, polyethylenes, or neoprenes. It is also readily understood that the lateral side portion and the medial side portion can be made from different types of materials provided that the materials can be suitable joined and further provide the desired density differential. According to embodiments, the density differential provides an increased pressure on the lateral half of the foot that shifts the ground reaction force vector more toward the knee-joint centre that can reduce the force the pushes the knee outward into a bow-legged posture.

According to embodiments, the lateral side portion and the medial side portion of the orthosis join at an oblique angle which defines a density transition region, for example a density transition region. It will be understood that a 45 degree angle will essentially create a density transition region having a length substantially equivalate to the thickness of the orthosis. An oblique angle larger than 45 degrees will substantially reduce this density transition region, while an oblique angle smaller thank 45 degree will increase the length of the density transition region. The oblique angle may be selected in order to adjust the degree of joint off-loading at the knee. Another reason for the variation of the oblique angle can include mitigation of the perception by a wearer of the density transition on the sole of their feet.

According to some embodiments, the oblique angle is selected such that the denser material is positioned on the bottom of the orthosis within the density transition region. In an alternate embodiment, the oblique angle is defined such that the denser material is positioned on the top of the orthosis within the density transition region.

In some embodiments, for example depending of a desired dose-response to the orthosis, the orthosis may include an external lateral wedge that is thicker on the lateral side of the foot and tapering down to the medial side. The angle of the lateral wedge can be between 3 and 5 degrees, however, other angles of the lateral wedge are possible, depending the desired dose/response and also possibly depending on lateral wedge thickness on the lateral side that can be accommodated within a shoe with which the orthosis is to be used. According to embodiments, the lateral wedge can support the variable density orthosis in aiding in the shifting of the ground reaction force vector to reduce the force compressing the medial compartment of the knee resulting in pain relief in individuals with osteoarthritis.

According to embodiments, it is considered that the orthosis according to embodiments of the present invention can improve upon existing over-the-counter arch supports by providing targeted pain relief to individuals with medial compartment knee osteoarthritis. Furthermore, reducing compartment load over a prolonged period has been shown to delay disease progression in people with knee osteoarthritis therefore potentially lowering the disease burden and socioeconomic costs associated with knee OA.

According to some embodiments, the variable density orthosis according to embodiments of the present invention may be integrated into the footwear by manufactures or may be integrated into the design of sock liners for footwear.

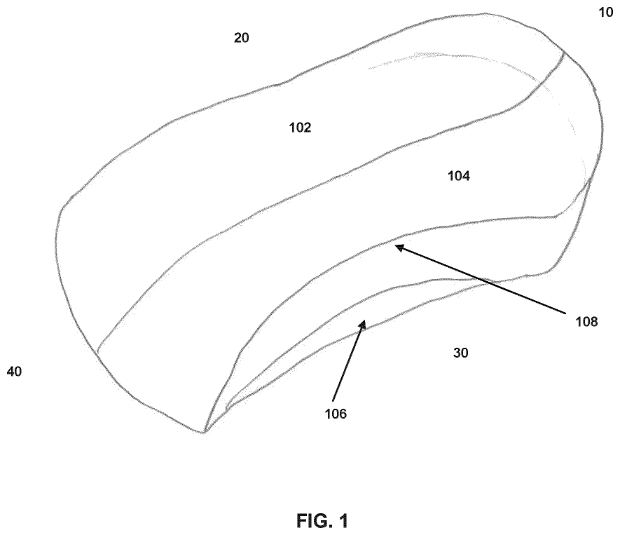

FIG. 1 illustrates a perspective view of a variable density orthosis for a right foot according to embodiments of the present invention. A lateral side portion 102 of the orthosis is defined along a length of the insole from a posterior end 10 to an anterior end 40 extending to the lateral side 20 of the orthosis. A medial side portion 104 of the orthosis is also defined along a length of the insole from a posterior end 10 to an anterior end 40 extending to the medial side 30 of the orthosis. On the medial side 30 of the orthosis there is provided a contoured arch support 108. In some embodiments, the volume 106 defined by the contoured arch support 108 can be filled with a filler in order to provide addition support to the arch of the foot of the user of the orthosis.

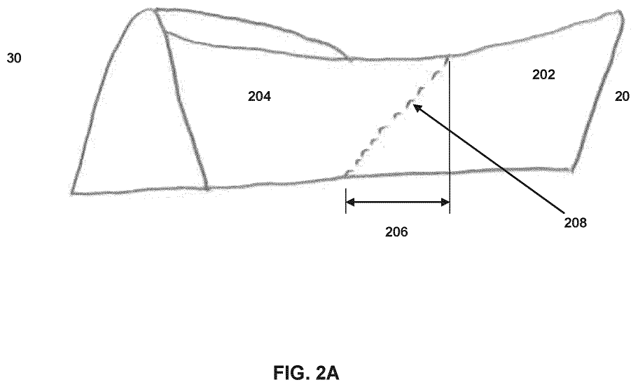

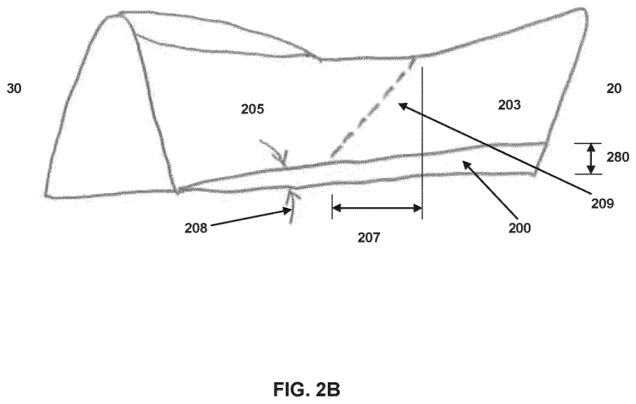

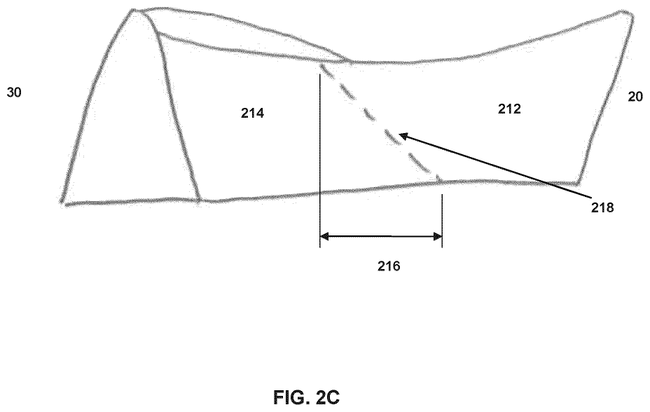

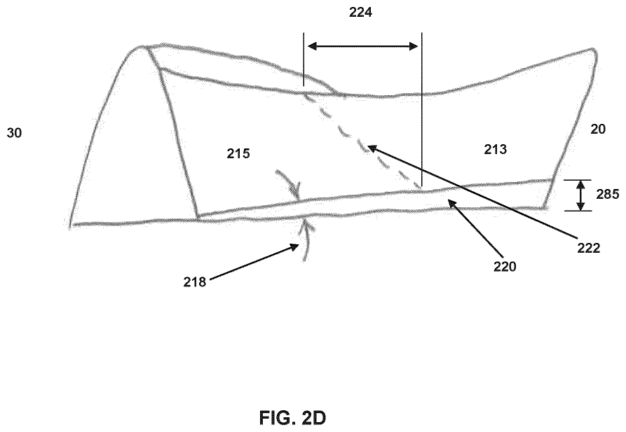

FIG. 2A illustrates a posterior view of a first embodiment of the variable density orthosis illustrated in FIG. 1. In this embodiment, the lateral side portion 202 is formed from a material that is denser than the medial side portion 204. The lateral side portion 202 and the medial side portion 204 join along an oblique angle defining the plane of connection 208. Depending on the oblique angle the length of the density transition region 206. It is noted that in this embodiment the denser material of the lateral side portion is below the material of the medial side portion.

FIG. 2B illustrates a posterior view of a second embodiment of the variable density orthosis illustrated in FIG. 1. In this embodiment is similar to that as illustrated in FIG. 2A, wherein the lateral side portion 203 is formed from a material that is denser than the medial side portion 205. The lateral side portion 203 and the medial side portion 205 join along an oblique angle defining the plane of connection 208. Depending on the oblique angle the length of the density transition region 207. This embodiment further includes a lateral wedge 200 which is attached to the bottom of the insole, and the lateral wedge can have an angle 208 which defines the opposite side height 280 of the lateral wedge.

FIG. 2C illustrates a posterior view of a third embodiment of the variable density orthosis illustrated in FIG. 1. In this embodiment, the lateral side portion 212 is formed from a material that is denser than the medial side portion 214. The lateral side portion 212 and the medial side portion 214 join along an oblique angle defining the plane of connection 218. Depending on the oblique angle the length of the density transition region 216. It is noted that in this embodiment the denser material of the lateral side portion is above the material of the medial side portion.

FIG. 2D illustrates a posterior view of a fourth embodiment of the variable density orthosis illustrated in FIG. 1. In this embodiment is similar to that as illustrated in FIG. 2C, wherein the lateral side portion 213 is formed from a material that is denser than the medial side portion 215. The lateral side portion 213 and the medial side portion 215 join along an oblique angle defining the plane of connection 222. Depending on the oblique angle the length of the density transition region 224. This embodiment further includes a lateral wedge 220 which is attached to the bottom of the insole, and the lateral wedge can have an angle 218 which defines the opposite side height 285 of the lateral wedge.

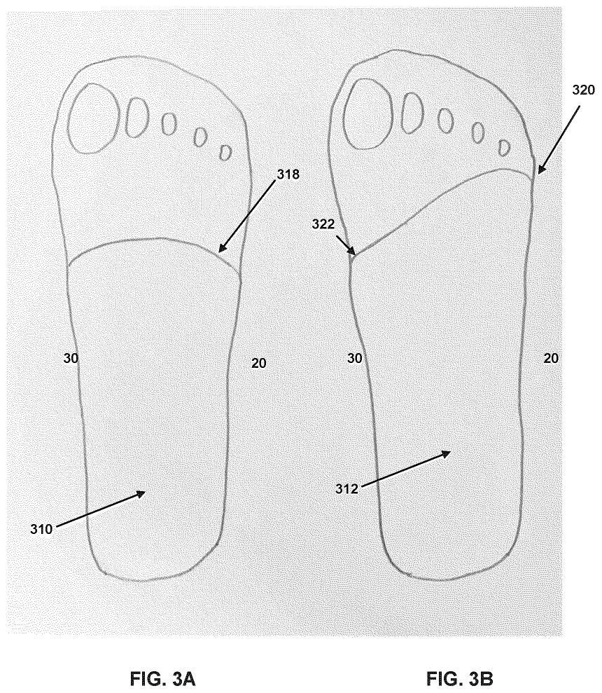

According to some embodiments, the variable density orthosis further includes a contouring shell attached to the top surface thereof. In some embodiments, as illustrated in FIG. 3A the contoured shell 310 is positioned and configured on the variable density orthosis as would be typical for an orthosis currently manufactured. For example, as illustrated in FIG. 3A the contoured shell extends to a location 318 approximately located at a position proximal to the 1st metatarsal head. According to other embodiments, as illustrated in FIG. 3B the contoured shell 312 is configured to extend distally to a predefined location, for example a location past the 5th metatarsal head 320, and then angling back/proximally to the standard shell position just proximal to the 1st metatarsal head 322. The inclusion of a contoured shell can provide a means for augmenting the effect of the variable density orthosis optionally with a lateral wedge. In these embodiments, the contoured shell can induce an out-toeing gait that may further reduce the knee adduction moment providing a further therapeutic relief to the medial compartment of the knee joint.

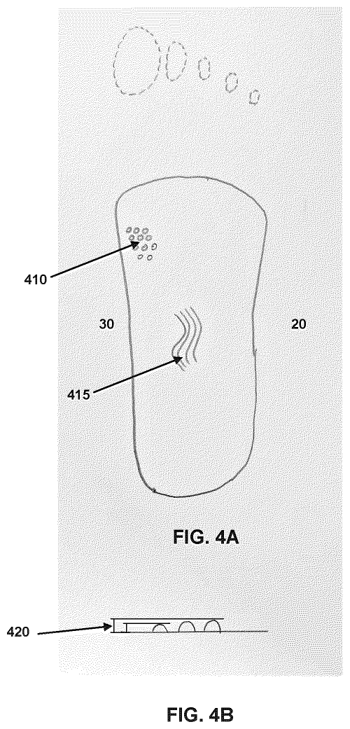

According to some embodiments, the variable density orthosis can further include a texture formed on the top surface thereof. The textured top surface can be used to enhance the postural stability of the user by increasing the cutaneous sensation on the plantar side of the foot. FIG. 4A illustrates two example of the texture that can be formed within the top surface and can include dimples 410 or waves 415 or other suitable texture as would be readily understood. These textured regions can be formed across the entire top surface of the variable density orthosis or at one or more selected regions of the top of the variable density orthosis. According to some embodiments, the selected region over which the texture is formed can be determined by one or more of a variety of desired secondary effects. These secondary effects can include increasing or decreasing pressure on one or more selected regions on the plantar side of the foot, stimulating sensation in one or more selected regions of the plantar side of the foot, or other secondary effects as would be readily understood by a worker skilled in the art. According to embodiments, as illustrated in FIG. 4B, the pattern can be formed such that the texture has a desired height 420, for example approximately 1 mm. This height can be dependent on the desired secondary effect or other features as would be readily understood. It is further readily understood that the height of the texture can be consistent over the one or more selected regions, or may vary in a desired manner over a selected region or vary between selected regions when texture is provide over multiple regions of the variable density orthosis. According to embodiments the texture can be formed using a soft or hard durometer material, and this type of material can be determined based on a desired effect for the inclusion of the texture. For example, the texture can be formed using a material that may include ethyl vinyl acetate, plastizote, polypropylene terephthalate (PPT) or other material as would be readily understood.

Based on the above discussion of an orthosis for the right foot in accordance with embodiments of the present invention, it would be readily understood by a worker skilled in the art how to configure an orthosis for the left foot in accordance with embodiments of the present invention.

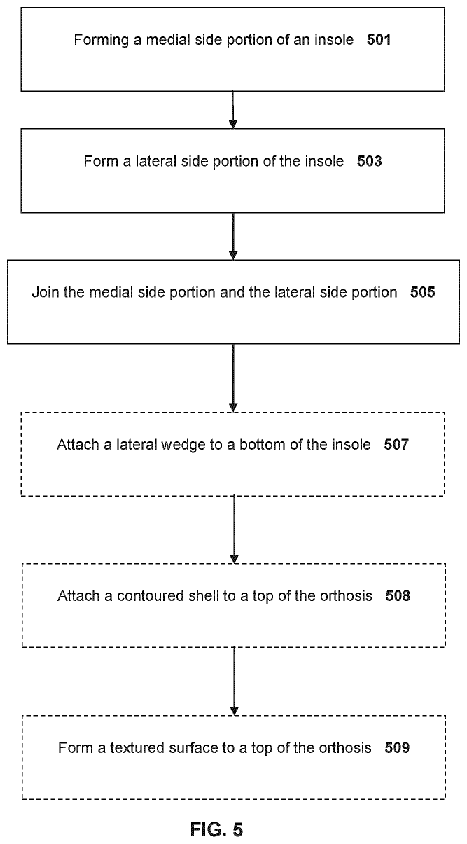

FIG. 5 illustrates a method an orthosis for alleviating osteoarthritis according to embodiments of the present invention. The method includes forming 501 a medial side portion of an insole from a first material having a first density, wherein the medial side portion, the medial side portion further having a contoured arch support portion. The method further includes forming 503 a lateral side portion of an insole from a second material having a second density, the second density greater than the first density. In addition, the method includes joining 505 the medial side portion and the lateral side portion, wherein the medial side portion and the lateral side portion join at an oblique angle along a length of the insole.

In some embodiments, the method further includes attaching 507 a lateral wedge to a bottom of the insole, the lateral wedge having an opposite side thereof positioned at a lateral side of the orthosis. In some embodiments, the method further includes attaching 508 a contoured shell to a top side of the orthosis. In some embodiments, the method further includes forming 509 or attaching a textured surface to a top side of the orthosis.

In some embodiments, forming one or more of the medial side portion, the lateral side portion and the lateral wedge is performed using three dimensional printing.

It will be appreciated that, although specific embodiments of the technology have been described herein for purposes of illustration, various modifications may be made without departing from the scope of the technology. The specification and drawings are, accordingly, to be regarded simply as an illustration of the invention as defined by the appended claims, and are contemplated to cover any and all modifications, variations, combinations or equivalents that fall within the scope of the present invention.

It is obvious that the foregoing embodiments of the invention are examples and can be varied in many ways. Such present or future variations are not to be regarded as a departure from the spirit and scope of the invention, and all such modifications as would be obvious to one skilled in the art are intended to be included within the scope of the following claims.

1. An orthosis comprising:
an insole having a lateral side portion and a medial side portion, the medial side portion formed from a first material having a first density and the lateral side portion formed from a second material having a second density, the second density greater than the first density, wherein the medial side portion and the lateral side portion join at an oblique angle, the medial side portion of the insole having a contoured arch support portion.

2. The orthosis according to claim 1 further comprising a lateral wedge coupled to a bottom of the insole, the lateral wedge having an opposite side thereof positioned at a lateral side of the orthosis.

3. The orthosis according to claim 1 further comprising an arch support material infilling an arch volume formed by the contoured arch support portion.

4. The orthosis according to claim 1 further comprising a contoured lateral portion and a contoured posterior portion.

5. The orthosis according to claim 1, further comprising a contoured shell, wherein the contoured shell extends distally to a location past a fifth metatarsal head.

6. The orthosis according to claim 1, further comprising a textured surface over one or more regions of the orthosis.

7. The orthosis according to claim 6, wherein the one or more regions are determined at least in part on one or more of increasing pressure on a selected region of a plantar side of a foot, decreasing pressure on a selected region of a plantar side of a foot and stimulating sensation in a selected region of a plantar side of a foot.

8. The orthosis according to claim 1, wherein the orthosis is used for alleviating knee osteoarthritis.

9. A method for making an orthosis, the method comprising:
forming a medial side portion of an insole from a first material having a first density, the medial side portion further having a contoured arch support portion;
forming a lateral side portion of an insole from a second material having a second density, the second density greater than the first density;
joining the medial side portion and the lateral side portion, wherein the medial side portion and the lateral side portion join at an oblique angle along a length of the insole.

10. The method according to claim 9 further comprising attaching a lateral wedge to a bottom of the insole, the lateral wedge having an opposite side thereof positioned at a lateral side of the orthosis.

11. The method according to claim 9, further comprising attaching a contoured shell, wherein the contoured shell extends distally to a location past a fifth metatarsal head.

12. The method according to claim 9, further comprising forming a textured surface over one or more regions of a top surface the orthosis.

13. The method according to claim 9, wherein forming one or more of the medial side portion and the lateral side portion is performed using three dimensional printing.

14. The method according to claim 9, wherein the method includes forming the orthosis to foot of a person suffering from knee osteoarthritis.
